(19) United States
(12) Patent Application Publication
(10) Pub. No.: US 2023/0268054 A1
(43) Pub. Date: Aug. 24, 2023

(54) AI ENABLED TREATMENT FOR CYBERCHONDRIA

(71) Applicant: International Business Machines Corporation, Armonk, NY (US)

(72) Inventors: Sri Harsha Varada, Vizianagaram (IN); Saraswathi Sailaja Perumalla, Visakhapatnam (IN); Yaswanth Konathala, Tyngsborough, MA (US); Kumar Gajula, Macherla (IN)

(21) Appl. No.: 17/677,273

(22) Filed: Feb. 22, 2022

(51) Int. Cl. G16H 20/70; G16H 50/20; G16H 50/30; G16H 40/20

(52) U.S. Cl. CPC G16H 20/70; G16H 50/20; G16H 50/30; G16H 40/20

(57) ABSTRACT

An approach for mitigating the impact of cyberchondria is disclosed. The approach includes retrieving user data, determining the context of the user, generating a user impact summary. Furthermore, the approach includes transmitting the user impact summary to a healthcare provider. The healthcare provider would make a diagnosis to the user based on the user impact summary and provides recommendations. The recommendations from the doctor can include scheduling an appointment with a mental health professional and/or treatment of the actual medical condition.

### BACKGROUND

The present invention relates generally to the field of healthcare, and more particularly to leveraging AI for treatment of cyberchondria.

Cyberchondria is a clinical phenomenon in which users perform repeated internet searches regarding medical information/symptoms/diagnosis that may impact their health. The repeated searches may be considered as excessive and too much concern about the physical health. It has been shown that cyberchondria is positively associated with symptoms of health anxiety.

Repeated internet search on symptoms or prescription is not a healthy practice, but as the technology has grown, users tend to use it more often. Due to time constraints, users will tend to take internet search help instead visiting a doctor or consult a health practitioner. Such practices influence, impact mental state of the patient and he or she may start assuming that they are suffering with major illness and actual situation may be a small health disturbance which can be treated with simple diet and minor exercises. For example, a user start searching for “headache” but end up with results for “brain tumor.”

### SUMMARY

Aspects of the present invention disclose a computer-implemented method, a computer system and computer program product for AI enabled treatment of cyberchondria. The computer implemented method may be implemented by one or more computer processors and may include, retrieving user data, determining context of the user, creating a user impact summary, communicating with healthcare practitioner based on the user impact summary, receiving doctor data packet from healthcare practitioner and executing the doctor data packet.

According to another embodiment of the present invention, there is provided a computer system. The computer system comprises a processing unit; and a memory coupled to the processing unit and storing instructions thereon. The instructions, when executed by the processing unit, perform acts of the method according to the embodiment of the present invention.

According to a yet further embodiment of the present invention, there is provided a computer program product being tangibly stored on a non-transient machine-readable medium and comprising machine-executable instructions. The instructions, when executed on a device, cause the device to perform acts of the method according to the embodiment of the present invention.

### BRIEF DESCRIPTION OF THE DRAWINGS

Preferred embodiments of the present invention will now be described, by way of example only, with reference to the following drawings, in which:

FIG. 1 is a functional block diagram illustrating a cyberchondria mitigation environment, designated as 100, in accordance with an embodiment of the present invention;

FIG. 2 is a functional block diagram representing the operation of health component 111, which includes inputs and outputs, in accordance with an embodiment of the present invention;

FIG. 3 is a high-level flowchart illustrating the operation of health component 111, designated as 300, in accordance with another embodiment of the present invention; and

FIG. 4 depicts a block diagram, designated as 400, of components of a server computer capable of executing the health component 111 within the cyberchondria mitigation environment 100, in accordance with an embodiment of the present invention.

### DETAILED DESCRIPTION

The current state of art as it pertains treatment of cyberchondria, can present some challenges. One challenge, for example, existing mitigation system may only focus on the escalation level of events associated with information searching and browsing of symptoms. Thus, it may not consider other contexts related to the current history of the users.

Embodiments of the present invention recognizes the deficiencies in the current state of art as it relates to assist users in managing cyberchondria and provides an AI (artificial intelligence) approach. One approach comprises utilizing a holistic picture of the user instead of focusing on internet search history. For example, the approach may leverage digital footprint data (besides internet page analytics), such as, from family and friend's interaction, gaze analytics, voice modulation, chat conversation, attention time and voice interactions. The data is digitized via a digital analysis module and sent to medical practitioner for a proper diagnosis.

Other embodiments may extend the prior embodiment but also include additional few steps. For example, one step is allowing the doctor to interact with the digital analysis module (a subcomponent of the health component 111, see digital analysis module 122) of the patient and comment/respond to the high cyberchondria impact generators for the patient to build more confidence and clarity and guide the patient accordingly. Another step is feeding a derived user impact summary (summary related to patient cyberchondria), to the digital analysis module which would give the suggestions and auto trigger next steps for patient. A final step is sharing the patient/family members user impact summary to corporate counselling services on demand and arrange a counselling session to so then the patient/family members thought process (i.e., to help avoiding any unwanted or irrelevant assumption).

Other embodiments may include steps additional steps, such as, i) capturing the user search words, blogs read and page navigation and portion of the page being highlighted or attention span of the user on a particular topic, gaze analysis and are measured and accordingly understand the context of search and summary of the search content and tag them to the digital analysis module, ii) analyzing the extracted patient chat conversation regarding health condition and use the information and tag them to digital analysis module, iii) understanding the change in user's voice modulation post search web content and tag that to the digital analysis module and iv) adjusting the digital analysis module of the patient, post a medical surgery or treatment and tries to self-interpret the medication dosage and health improvement or deterioration by assuming false symptoms.

References in the specification to “one embodiment”, “an embodiment”, “an example embodiment”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments, whether or not explicitly described.

It should be understood that the Figures are merely schematic and are not drawn to scale. It should also be understood that the same reference numerals are used throughout the Figures to indicate the same or similar parts.

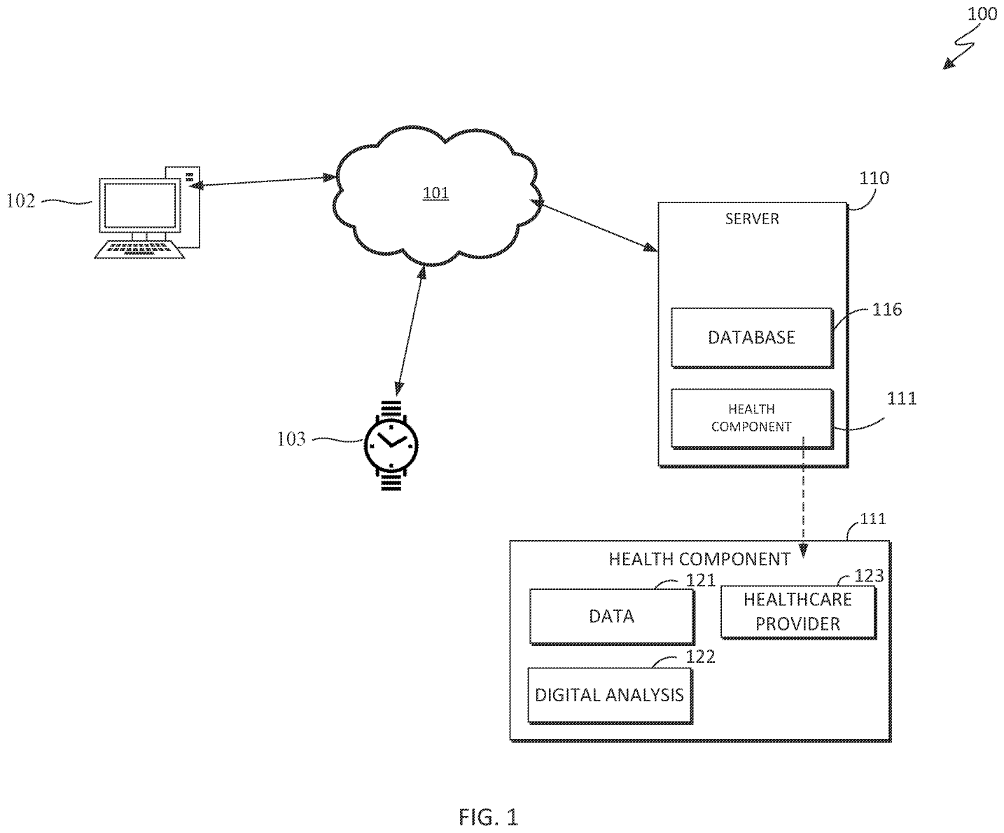

FIG. 1 is a functional block diagram illustrating an edge environment, designated as 100, in accordance with an embodiment of the present invention. FIG. 1 provides only an illustration of one implementation and does not imply any limitations with regard to the environments in which different embodiments may be implemented. Many modifications to the depicted environment may be made by those skilled in the art without departing from the scope of the invention as recited by the claims.

Cyberchondria mitigation environment 100 includes network 101, client device 102, and healthcare device 103.

Network 101 can be, for example, a telecommunications network, a local area network (LAN), a wide area network (WAN), such as the Internet, or a combination of the three, and can include wired, wireless, or fiber optic connections. Network 101 can include one or more wired and/or wireless networks that are capable of receiving and transmitting data, voice, and/or video signals, including multimedia signals that include voice, data, and video information. In general, network 101 can be any combination of connections and protocols that can support communications between server 110, client device 102, healthcare device 103 and other computing devices (not shown) within cyberchondria mitigation environment 100. It is noted that other computing devices can include, but is not limited to, client devices 102 and any electromechanical devices capable of carrying out a series of computing instructions.

Client device 102 is a computing device belonging to the user. Client device 102 has internet connection and ability to send/retrieve information from online data sources. Client device 102 can be used to facilitate real-time conversation (e.g., text or voice) with another person via chat software. For example, client device 102 is a laptop computer that user frequently uses to access the internet.

Healthcare device 103 is a smart device (e.g., smart phone, IoT device, smart watch) capable of recording/measuring physical phenomena and/or characteristic of the user or relating to the user. For example, healthcare device 103 can be a smartwatch that the user wears to monitor various user vital statistics (e.g., heart rate, blood pressure, etc.).

Embodiment of the present invention can reside on server 110, client device 102 or healthcare device 103. Server 110 includes health component 111 and database 116.

Server 110 can be a standalone computing device, a management server, a web server, a mobile computing device, or any other electronic device or computing system capable of receiving, sending, and processing data. In other embodiments, server 110 can represent a server computing system utilizing multiple computers as a server system, such as in a cloud computing environment. In another embodiment, server 110 can be a laptop computer, a tablet computer, a netbook computer, a personal computer (PC), a desktop computer, a personal digital assistant (PDA), a smart phone, or any other programmable electronic device capable of communicating other computing devices (not shown) within cyberchondria mitigation environment 100 via network 101. In another embodiment, server 110 represents a computing system utilizing clustered computers and components (e.g., database server computers, application server computers, etc.) that act as a single pool of seamless resources when accessed within cyberchondria mitigation environment 100.

Health component 111 provides a holistic capability of, but is not limited to, i) analyzing patient/family member's internet search in context of symptoms or prescription, blogs read, text had highlighted while reading the blog and cyberchondria impact analysis (i.e., from chat/blogs/internet search), ii) share digital analysis content holding patient cyberchondria impact to doctor in next visit so that he/she can give better advice and can provide right counselling to clear any specific concerns or for any dubiety, ii) suggest patient to take online consultation with other doctor for clarifying confusion or worries he/she had, iii) sharing that to corporate counselling services and arrange counselling sessions as required and iv) allowing doctor to create a patient actual state/condition as data packet and injecting to patient internet search, so that internet can list those results whose objective is closer to doctor data packet. Additionally, those results (from (iv)), which have similar objective of doctor data packet, can have additional icon for differentiation. Doctor data packets refers to, the patient exact health/mental state condition and prescription and associated reports in medical terminology. Essentially, a mental note by the doctor regarding the prognosis of the patient (patient may not understand the relevancy at the time) but which can be tagged to that patient. Furthermore, doctor data packet can contain recommendation and next steps for treatment of the patient. The capability of health component 111 can include, vi) suggesting doctors or a world health organization (WHO) to create digital footprints like blogs or videos by taking a few points into consideration (e.g., the all the data packets from different doctors for different patients and digital analysis and cyberchondria impacts of different patients), vii) a governing body which can approve any blog written by outsider with appropriate data packet and objective mapping to associated cyberchondria impact and viii) define a renewal time for content created WHO (from list (vi)) that would help future generations learn the knowledge corpus without losing the truthiness expiry over time.

Health component 111 can include the following subcomponents (i.e., modules), data module 121, digital analysis module 122 and healthcare provider module 123.

As is further described herein below, data module 121 of the present invention provides the capability of interfacing with various data sources (e.g., internet search history of computing devices belonging to the user, IoT devices that is situated around the user, etc.) to retrieve/scour for relevant patient data information related to the user. It is noted that existing techniques for capturing user search history pattern, scouring user digital trails and search query patterns can be used by data module 121. Patient data information can include, user internet search history, voice history/pattern (i.e., from IoT devices), gaze analysis and medical records of the user. For example, data module 121 captures the user search words, blogs read and page navigation and portion of the page being highlighted or attention span of the user on a particular topic, gaze analysis and are measured and accordingly, tries to understand the context of search and summary of the search content and forwards them to the digital analysis module 122. In another example, data module 121 may extracted patient chat conversation regarding health condition to be used for digital analysis module 122. In yet another example, data module 121 may observe internet post by the user about a medical surgery or treatment and tries to self-interpret the medication dosage and health improvement or deterioration.

As is further described herein below, digital analysis module 122 of the present invention provides the capability of utilizing patient data information and determining solutions to mitigate the effect of cyberchondria (via machine learning), by creating one or more user impact summaries. It is noted that any machine learning techniques may be used to analyze user data, predicting outcomes of behavior/actions of the user based on the user data and determining solutions based on, but it is not limited to, available user data, recommendations by health provider, medical data sources, etc. Furthermore, user has the option of consenting or not to the system to retrieve/analyze data related to the user. In other embodiment, digital analysis module 122, determines the impact of cyberchondria on a user and on how much it has affected the user (i.e., is the user carried away with the search, etc.).

How does digital analysis module 122 understand and/or determine the impact on the user? This can be done by analyzing patient data information and creating a user impact summary. A user impact summary is a “report” associated with the user as it relates to their excessive searching on the internet for medical information (e.g., symptoms, diseases, treatment, etc.) associated with their own health (which could be perfectly healthy). The user impact summary can include a ranking or scoring methodology to indicate whether the user is at risk of having cyberchondria. The ranking or scoring methodology can be as simple as, “Low-No Risk”, “Medium-Risk” or “High-Risk”. The user impact summary may include other data to support the ranking conclusion, such as, internet search data, chat data, conversation data and phone call data). For example, if there is a change in voice pattern to indicate the user is always in a somber mood and speaks in a low monotonous manner (i.e., indicating sadness) then the system can recognize that cyberchondria has a profound affect (and possibly adverse effect) on the user (i.e., user impact summary would denote a score/ranking of “High-Risk”). Thus, based on the user impact summary, the system could suggest a visit to a medical provider (via healthcare provider module 123).

What solutions or suggestions can digital analysis module 122 provide? Depending on the severity of the impact by cyberchondria on the user (user impact summary), digital analysis module 122 could provide the following, but it is not limited to, i) suggesting (via a generated avatar) the patient to visit the doctor to avoid confusion and would book appropriate doctor appointment, ii) sharing the patient/family members cyberchondria impact to corporate counselling services on demand and arrange a counselling session to soothe the patient/family members mental state, iii) dynamically predict (via machine learning) that the patient might exhibit an early sign of cyberchondria and suggest a doctor visit and iv) receiving doctor data packet (i.e., recommendation) from the healthcare provider and complying with the recommendation from the healthcare provider. Doctor data packets refers to, the patient exact health/mental state condition and prescription and associated reports in medical terminology. Essentially, a mental note by the doctor regarding the prognosis of the patient (patient may not understand the relevancy at the time) but which can be tagged to that patient. The tagged information can be used by search engines to show more tailored results that are applicable to patient. Furthermore, doctor data packet can contain recommendation and next steps for treatment of the patient.

Other actions or functionality provided by digital analysis module 122 can include, receiving the data input (“user impact summaries” and doctor data packet (if available)) and based on that, the system would map that impact to a known/prefeed psychology/emotional models which will help to pick the appropriate next steps/suggestions for the patient. For example, patient 1 is starting to worry too much about the covid symptoms, but the doctor data packet says its mild stage 1. Thus, digital analysis module 122, would suggest to stay isolated and show some videos of the happy discharged covid patients. And share suggestions as, “Talk to your family”, “Have fun games online” etc.

In another example, Patient 2 started to worry about the dengue symptoms (but no doctor data packet available), digital analysis module 122 would suggest the user not to panic but instead book an online doctor appointment. System may provide links to some articles referring the diagnosis as something else not related to dengue and suggesting few precautionary steps that need to be taken, even before consulting/doctor or before getting the test.

Other actions or functionality provided by digital analysis module 122 can include optimizing search engine that can be applied on demand for patient/relatives/friends that wants to learn the results that mapping of the patient exact health condition (i.e., to avoid unnecessary over head or confusion to patients and relatives) based on doctor data packet and user impact summary. For example, a search engine would show a different flag/icon against the results that are mapped with doctor data packet and indicating the percentage match.

As is further described herein below, healthcare provider module 123 of the present invention provides the capability of coordinating and managing the results of user impact summary and communicating that information to/with a healthcare provider. Additionally, healthcare provider may transmit recommendations (treatment for cyberchondria) back to healthcare provider module 123.

Other functionality of healthcare provider module 123 may include, i) enabling doctors to feed the search engines with patient identity and the data packet that holds patient exact health condition through medical terminology and current state of patient and recovery techniques, ii) allowing the doctor to interact with the digital analysis module of the patient and comment/respond to the high cyberchondria impact generators for the patient to build more confidence and clarity and guide the patient accordingly, iii) generating an avatar that explains the cyberchondria impact of patient recorded in the digital analysis module with related background activities to doctor for quick catch up and iv) creating a centralized system that would take doctor data packets from all the doctors for different types of patient(s)/their mental state(s)/their health condition(s) and crawl the web with available resources and identify the digital footprint delta to suggest governing body (i.e., WHO) to enable required resources (i.e., centralized system would also define the renewal time for available digital foot print data so as to help future generations to get knowledge corpus without losing the truthiness expiry over time). A digital footprint data can be digital information related to the user, such as, different blogs, videos, papers, articles that are available in web which are generally referred by users by searching their own symptom/diseases and medicines prescription.

Database 116 is a repository for data used by health component 111. Database 116 can be implemented with any type of storage device capable of storing data and configuration files that can be accessed and utilized by server 110, such as a database server, a hard disk drive, or a flash memory. Database 116 uses one or more of a plurality of techniques known in the art to store a plurality of information. In the depicted embodiment, database 116 resides on server 110. In another embodiment, database 116 may reside elsewhere within cyberchondria mitigation environment 100, provided that health component 111 has access to database 116. Database 116 may store information associated with, but is not limited to, knowledge corpus of, user medical history, user observation/measurements from IoT devices (i.e., 103), user activities (e.g., online or physical), doctor data packet, digital footprint data and medical records of the user.

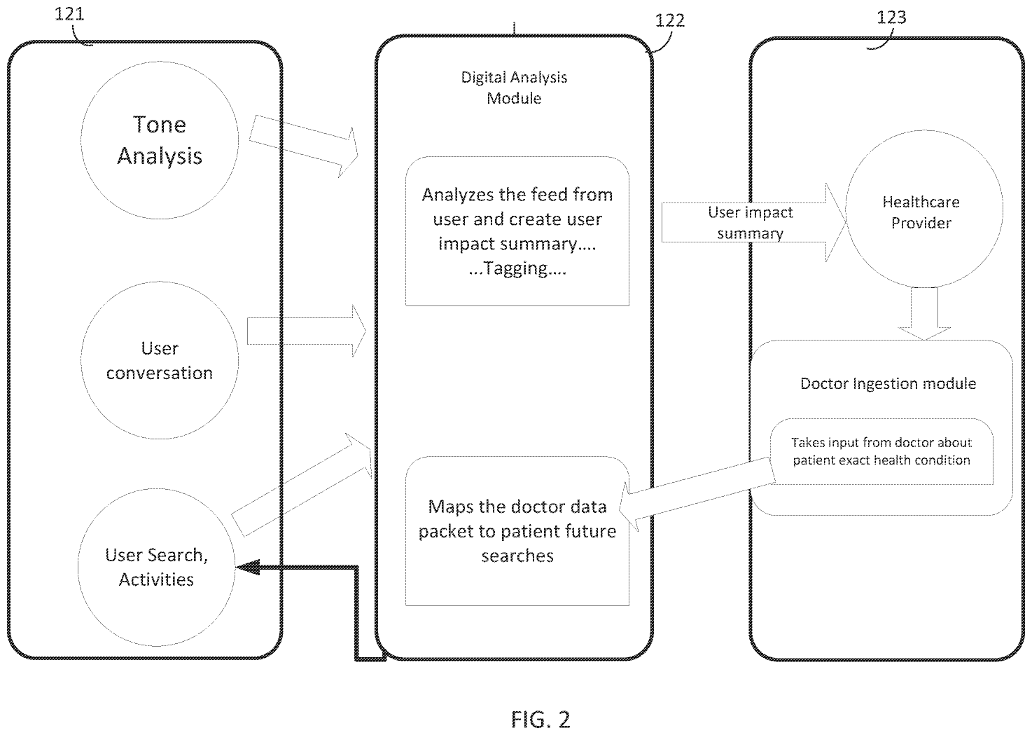

FIG. 2 is a functional block diagram representing the interaction between subcomponents (e.g., data module 121, digital analysis module 122 and healthcare provider module 123) health component 111, which includes inputs and outputs. For example, data module 121 retrieves user data related to the user, such as, digital conversation of the user (i.e., chats), internet search and internet activity of the user and voice (i.e., tone) data-based conversation of the user and others and transmits the data to digital analysis module 122. Digital analysis module 122 analyzes the combined data and determines/identifies context of the user (as it relates to healthcare diagnosis). The context of the user relates to the reasoning for the user to incessant search the internet for medical information. Digital analysis module 122 creates a user impact summary and transmit it to healthcare provider module 123. Furthermore, digital analysis module 122 can tag metadata to the data of the user. Healthcare provider module 123 relays the information to a healthcare provider and receives recommendations from healthcare provider (i.e., doctors) and transmit that result back to digital analysis module 122 to be mapped to patient's future internet search (eventually that mapping will be part of history data/synopsis of the internet search and activities of the user).

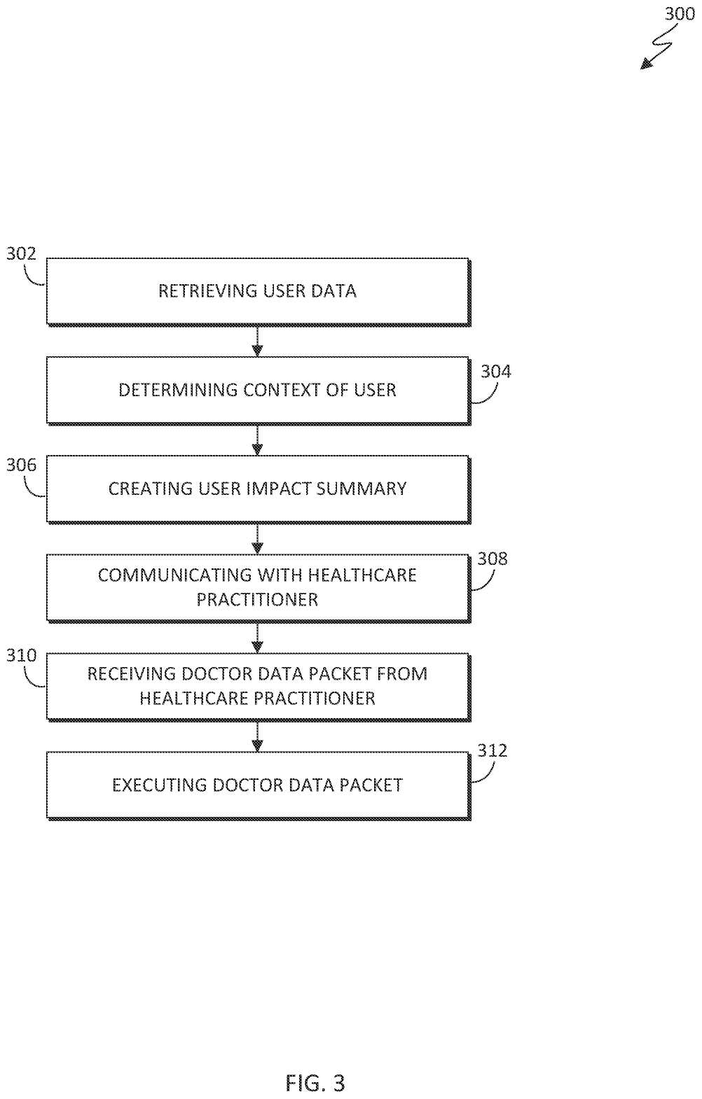

FIG. 3 is a high-level flowchart illustrating the operation of health component 111, designated as 300, in accordance with an embodiment of the present invention.

Health component 111 retrieves user data (step 302). In an embodiment, health component 111, through data module 121, retrieves data from various data sources relating to the user. User data can include, but it is not limited to, tonal data, user conversation user internet search and internet activities. An example scenario will be presented to accompany the process steps, user_1 is 55 years old and is not in the best of health. A medical practitioner may even state that user_1 might be considered immune-compromised. User_1 has just developed a cough and is worried that it might be COVID-19 or another major disease. User_1 has been searching the internet for symptoms related to COVID-19 or symptoms related to the cough. Data module 121 can search the internet search history of user_1 and retrieve other data (e.g., voice, blood pressure, heart rate, etc.) from healthcare device 103.

Health component 111 determines the context of the user (step 304). In an embodiment, health component 111, through digital analysis module 122, understands the context of the data (e.g., search, summary of search content, etc.) of the user and tags the data to digital analysis module 122. For example, understanding the context of the data means that based on user search history pattern and search query patterns and navigation aspects are considered to understand/determine the user's goal (i.e., intent/reasons) in performing searches for long hours or during abnormal hours (i.e., late hours) or always keep searching some health related data. Tagging means that health component 111 has mapped/assigning metadata to the user's data record. The metadata can summarize the intent/goal of the user data. Referring to the previous sample scenario, digital analysis module 122 observes that user_1 is incessantly searching for symptoms related to coughs and possible diseases related to coughing. Thus, digital analysis module 122 is able to determine the intent/goal of user_1 (i.e., worried about COVID as the main cause of the cough) based on the internet search history and chat conversations between user_1 and his family. Based on the intent/reasons, digital analysis module 122 tags the user data with metadata (i.e., summary of the intent/reasons of the user).

Health component 111 creates a user impact summary (step 306). In an embodiment, health component 111, through digital analysis module 122, creates a user impact summary. Recall that a user impact summary can include a ranking or scoring methodology to indicate whether the user is at risk of having cyberchondria. The ranking or scoring methodology can be as simple as, “Low-No Risk”, “Medium-Risk” or “High-Risk”. The user impact summary may include other data to support the ranking conclusion, such as, internet search data, chat data, conversation data and phone call data). Referring to the previous sample scenario, digital analysis module 122, generates a user impact summary for user_1 based on the intent of user_1. For example, the content of the user impact summary of user_1 states that user_1 is at “High-Risk” based on activities/history related to user_1. For example, user_1 is consumed with the finding out if the cough is related to COVID-19 or not, where it has made user_1 sad most of the time (e.g., based on chat conversation with family and tone change during web calls).

In another embodiment, after creating a user impact summary, health component 111 could suggest such as, but is not limited to, suggesting if there is a need for counselling or consulting a doctor at any stage thus avoids leading to cyberchondria.

Health component 111 communicates with healthcare practitioner (step 308). In an embodiment, health component 111, through digital analysis module 122 and/or healthcare provider module 123, transmits the user impact summary to a healthcare practitioner (see FIG. 2) along with any other data associated with the user. Other data can be shared during consultation of doctor, doctor could get glimpse of the background of user mental state and accordingly clarifies his queries or provides treatment. Referring to the previous sample scenario, health component 111, submits the user impact summary of user_1 to doctor_1 (i.e., primary care physician for user_1).

Health component 111 receives doctor data packet from the healthcare practitioner (step 310). In an embodiment, health component 111, receives one or more recommendations from the healthcare practitioner (as a doctor data packet) as it relates to the user. The recommendation can include a diagnosis that the user has cyberchondria and suggest that the user should schedule a treatment with a mental health professional. In an embodiment, health component 111, receives one or more recommendation from healthcare practitioner based on the user impact summary. The healthcare practitioner will analyze the user impact summary and make a diagnosis (i.e., user actually has a real medical issue or user is a borderline cyberchondriac). Same methodology can be implemented post any surgery or treatment for a patient and monitor his web usage behavior to consider his health improvement or recovery rate. Referring to the previous sample scenario, health component 111, receives recommendation from doctor_1 as it relates to user_1. For example, the recommendation is that the cough is normal and not associated with any risky disease and user_1 is at risk of cyberchondria and should see a mental health professional.

Health component 111 executes the recommendation (step 312). In an embodiment, health component 111, executes the one or more recommendation from the healthcare practitioner. If the recommendation is for the user to schedule an appointment with a mental health professional, then the system may make that appointment on behave of the user or could alert the user to make the appointment themselves. Referring to the previous sample scenario, the recommendation from doctor_1 is that user_1 should see a mental health professional due to having symptoms associated with cyberchondria, the system can automatically schedule an appointment with a mental health professional.

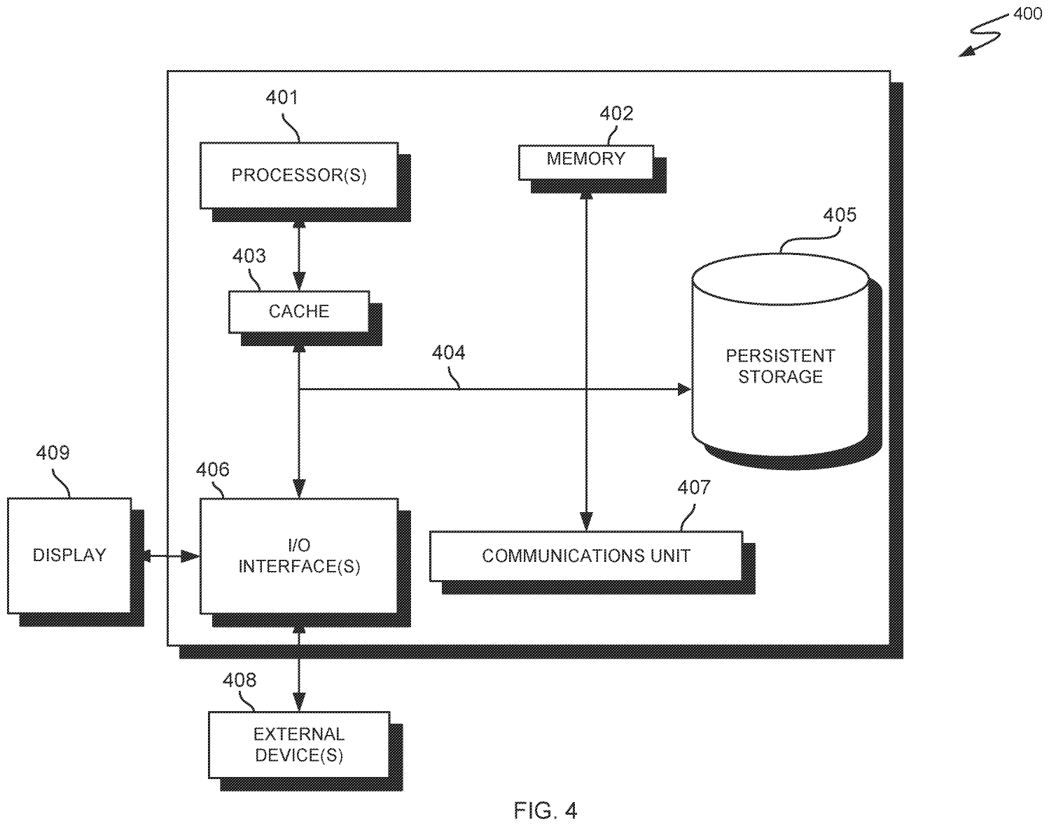

FIG. 4, designated as 400, depicts a block diagram of components of health component 111 application, in accordance with an illustrative embodiment of the present invention. It should be appreciated that FIG. 4 provides only an illustration of one implementation and does not imply any limitations with regard to the environments in which different embodiments may be implemented. Many modifications to the depicted environment may be made.

FIG. 4 includes processor(s) 401, cache 403, memory 402, persistent storage 405, communications unit 407, input/output (I/O) interface(s) 406, and communications fabric 404. Communications fabric 404 provides communications between cache 403, memory 402, persistent storage 405, communications unit 407, and input/output (I/O) interface(s) 406. Communications fabric 404 can be implemented with any architecture designed for passing data and/or control information between processors (such as microprocessors, communications and network processors, etc.), system memory, peripheral devices, and any other hardware components within a system. For example, communications fabric 404 can be implemented with one or more buses or a crossbar switch.

Memory 402 and persistent storage 405 are computer readable storage media. In this embodiment, memory 402 includes random access memory (RAM). In general, memory 402 can include any suitable volatile or non-volatile computer readable storage media. Cache 403 is a fast memory that enhances the performance of processor(s) 401 by holding recently accessed data, and data near recently accessed data, from memory 402.

Program instructions and data (e.g., software and data x10) used to practice embodiments of the present invention may be stored in persistent storage 405 and in memory 402 for execution by one or more of the respective processor(s) 401 via cache 403. In an embodiment, persistent storage 405 includes a magnetic hard disk drive. Alternatively, or in addition to a magnetic hard disk drive, persistent storage 405 can include a solid state hard drive, a semiconductor storage device, a read-only memory (ROM), an erasable programmable read-only memory (EPROM), a flash memory, or any other computer readable storage media that is capable of storing program instructions or digital information.

The media used by persistent storage 405 may also be removable. For example, a removable hard drive may be used for persistent storage 405. Other examples include optical and magnetic disks, thumb drives, and smart cards that are inserted into a drive for transfer onto another computer readable storage medium that is also part of persistent storage 405. Health component 111 can be stored in persistent storage 405 for access and/or execution by one or more of the respective processor(s) 401 via cache 403.

Communications unit 407, in these examples, provides for communications with other data processing systems or devices. In these examples, communications unit 407 includes one or more network interface cards. Communications unit 407 may provide communications through the use of either or both physical and wireless communications links. Program instructions and data (e.g., health component 111) used to practice embodiments of the present invention may be downloaded to persistent storage 405 through communications unit 407.

I/O interface(s) 406 allows for input and output of data with other devices that may be connected to each computer system. For example, I/O interface(s) 406 may provide a connection to external device(s) 408, such as a keyboard, a keypad, a touch screen, and/or some other suitable input device. External device(s) 408 can also include portable computer readable storage media, such as, for example, thumb drives, portable optical or magnetic disks, and memory cards. Program instructions and data (e.g., health component 111) used to practice embodiments of the present invention can be stored on such portable computer readable storage media and can be loaded onto persistent storage 405 via I/O interface(s) 406. I/O interface(s) 406 also connect to display 409.

Display 409 provides a mechanism to display data to a user and may be, for example, a computer monitor.

The programs described herein are identified based upon the application for which they are implemented in a specific embodiment of the invention. However, it should be appreciated that any particular program nomenclature herein is used merely for convenience, and thus the invention should not be limited to use solely in any specific application identified and/or implied by such nomenclature.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration but are not intended to be exhaustive or limited to the embodiments disclosed. Many modifications and variations will be apparent to those of ordinary skill in the art without departing from the scope and spirit of the invention. The terminology used herein was chosen to best explain the principles of the embodiment, the practical application or technical improvement over technologies found in the marketplace, or to enable others of ordinary skill in the art to understand the embodiments disclosed herein.

The corresponding structures, materials, acts, and equivalents of all means or steps plus function elements in the claims below are intended to include any structure, material, or act for performing the function in combination with other claimed elements, as specifically claimed. The description of the present invention has been presented for purposes of illustration and description, but is not intended to be exhaustive or limited to the invention in the form disclosed. Many modifications and variations will be apparent to those of ordinary skills in the art without departing from the scope and spirit of the invention. The embodiments are chosen and described in order to best explain the principles of the invention and the practical application, and to enable others of ordinary skills in the art to understand the invention for various embodiments with various modifications, as are suited to the particular use contemplated.

What is claimed is:

1. A computer-implemented method for mitigating the impact of cyberchondria, the computer-method comprising:
retrieving user data;
determining context of the user;
creating a user impact summary;
communicating with healthcare practitioner based on the user impact summary;
receiving doctor data packet from healthcare practitioner; and
executing the doctor data packet.

2. The computer-implemented method of claim 1, further comprising:
generating one or more initial recommendation for the user based on the user impact summary, wherein the one or more initial recommendation further comprises of, asking the user to schedule an appointment with a primary care physician.

3. The computer-implemented method of claim 1, wherein the user data comprises of web browsing history of the user, chat conversation of the user and phone conversation of the user.

4. The computer-implemented method of claim 1, wherein determining the context of the user further comprises of understanding the reason of the user as it relates to the user data.

5. The computer-implemented method of claim 1, wherein the user impact summary is a report associated with the effect of cyberchondria on the user and has a ranking of, low-no risk, medium-risk or high-risk.

6. The computer-implemented method of claim 1, further comprising:
tagging a metadata to the user data, wherein the metadata contains a summary of the mental state of the user.

7. The computer-implemented method of claim 1, wherein the doctor data packet comprises of a summary of the mental state of the user and recommendation for treatment for the user.

8. The computer-implemented method of claim 1, wherein executing the doctor data packet further comprises of, recommending the user seek help from a mental health professional.

9. A computer program product for mitigating the impact of cyberchondria, the computer program product comprising:
one or more computer readable storage media and program instructions stored on the one or more computer readable storage media, the program instructions comprising:
program instructions to retrieve user data;
program instructions to determine context of the user;
program instructions to create a user impact summary;
program instructions to communicate with healthcare practitioner based on the user impact summary;
program instructions to receive doctor data packet from healthcare practitioner; and
program instructions to execute the doctor data packet.

10. The computer program product of claim 9, further comprising:
program instructions to generate one or more initial recommendation for the user based on the user impact summary, wherein the one or more initial recommendation further comprises of, asking the user to schedule an appointment with a primary care physician.

11. The computer program product of claim 9, wherein the user data comprises of web browsing history of the user, chat conversation of the user and phone conversation of the user.

12. The computer program product of claim 9, wherein program instructions to determine the context of the user further comprises of understanding the reason of the user as it relates to the user data.

13. The computer program product of claim 9, wherein the user impact summary is a report associated with the effect of cyberchondria on the user and has a ranking of, low-no risk, medium-risk or high-risk.

14. The computer program product of claim 9, further comprising:
program instructions to tag a metadata to the user data, wherein the metadata contains a summary of the mental state of the user.

15. A computer system for mitigating the impact of cyberchondria, the computer system comprising:
one or more computer processors;
one or more computer readable storage media; and
program instructions stored on the one or more computer readable storage media for execution by at least one of the one or more computer processors, the program instructions comprising:
program instructions to retrieve user data;
program instructions to determine context of the user;
program instructions to create a user impact summary;
program instructions to communicate with healthcare practitioner based on the user impact summary;
program instructions to receive doctor data packet from healthcare practitioner; and
program instructions to execute the doctor data packet.

16. The computer system of claim 15, further comprising:
program instructions to generate one or more initial recommendation for the user based on the user impact summary, wherein the one or more initial recommendation further comprises of, asking the user to schedule an appointment with a primary care physician.

17. The computer system of claim 15, wherein the user data comprises of web browsing history of the user, chat conversation of the user and phone conversation of the user.

18. The computer system of claim 15, wherein program instructions to determine the context of the user further comprises of understanding the reason of the user as it relates to the user data.

19. The computer system of claim 15, wherein the user impact summary is a report associated with the effect of cyberchondria on the user and has a ranking of, low-no risk, medium-risk or high-risk.

20. The computer system of claim 15, further comprising:
program instructions to tag a metadata to the user data, wherein the metadata contains a summary of the mental state of the user.
